(19) United States
(12) Patent Application Publication
(10) Pub. No.: US 2009/0196918 A1
(43) Pub. Date: Aug. 6, 2009

(54) Liposomal formulations of hydrophobic lactone drugs in the presence of metal ions

(75) Inventors: Vijay JOGUPARTHI, Lexington, KY (US); Bradley D. Anderson, Lexington, KY (US)

Correspondence Address: CROWELL & MORING LLP;INTELLECTUAL PROPERTY GROUP, P.O. BOX 14300, WASHINGTON, DC, 20044-4300, US

(73) Assignee: University of Kentucky Research Foundation, Lexington, KY (US)

(21) Appl. No.: 12/363,420

(22) Filed: Jan. 30, 2009

Related U.S. Application Data
(60) Provisional application No. 61/025,601, filed on Feb. 1, 2008.

(51) Int. Cl. A61K 9/127; A61K 31/4375

(52) U.S. Cl. 424/450; 514/283

(57) ABSTRACT

Provided is a liposome comprising a hydrophobic lactone drug and a cyclodextrin, wherein the liposome has an intraliposomal pH and cyclodextrin concentration such that upon administration of the liposome to a subject, the liposome exhibits a uniform release profile of the hydrophobic lactone drug. Also provided is a method of administering a hydrophobic lactone drug to a subject in need thereof. The method comprises administering a liposome to the subject in need, wherein the liposome comprises the hydrophobic lactone drug and a cyclodextrin. The liposome has an intraliposomal pH and cyclodextrin concentration such that upon administration of the liposome to the subject, the liposome exhibits a uniform release profile of the hydrophobic lactone drug.

### CLAIM OF PRIORITY

This application claims priority under 35 U.S.C. § 119 to U.S. Provisional Application No. 61/025,653, filed Feb. 1, 2008, the entire contents of which are hereby incorporated by reference.

### STATEMENTS REGARDING FEDERALLY SPONSORED RESEARCH

The invention was funded in part by Grant Nos. R01 CA87061 awarded by the National Institutes of Health and National Cancer Institute. The government has certain rights in the invention.

### FIELD OF ART

The liposomes and their associated methods of administration disclosed herein relate to the delivery of pharmaceutically effective amounts of hydrophobic lactone drugs.

### BACKGROUND

Liposomes are spherical nanoparticles comprising one or more concentric lipid bilayers enclosing an aqueous interior. Liposomes with a single concentric bilayer have a typical size range of ˜50-200 nm and are referred to as unilamellar vesicles. Liposomes with more than one concentric bilayer are referred to as multilamellar vesicles.

Liposomes can release drugs to a target tissue or can release drugs in circulation. Unilamellar vesicles are of particular pharmaceutical use in targeting specific tissues in the body, such as the spleen or tumors.

Liposomes can be used to encapsulate both hydrophilic and hydrophobic drug molecules. Hydrophobic molecules are thought to partition into the lipid bilayer, thereby gaining protection against a variety of reactions which they are prone to in the aqueous phase. However, liposomes can also be used as carriers for hydrophilic molecules by entrapping these molecules in the aqueous core of liposomes.

Liposomes are particularly suited to the delivery of chemotherapeutic drugs to tumors. Encapsulation of chemotherapeutics in liposomes is advantageous because liposomes preferentially accumulate in tumors and can avoid exposing healthy tissue to the chemotherapeutics avoiding undesirable side effects. However, in order to target specific tissue, the liposomes must retain the entrapped drug while in circulation to allow sufficient time for accumulation of the liposomes in the target tissue. Upon accumulation in the target tissue, the liposomes must then release the entrapped drug.

A prominent class of chemotherapeutic drugs are camptothecins. Camptothecins fall within the larger class of hydrophobic lactone drugs.

There is a need for novel formulation techniques for improved liposomal loading, improved liposomal retention, and prolonged liposomal release of camptothecins and similar hydrophobic lactone drugs.

### SUMMARY

Disclosed herein is a liposome comprising a hydrophobic lactone drug and a cyclodextrin, wherein the liposome has an intraliposomal pH and a cyclodextrin concentration such that upon administration of the liposome to a subject, the liposome exhibits a uniform release profile of the hydrophobic lactone drug.

Also disclosed herein is a method of administering a hydrophobic lactone drug to a subject in need thereof, comprising administering a liposome to the subject in need, wherein the liposome comprises the hydrophobic lactone drug and a cyclodextrin, the liposome having an intraliposomal pH and a cyclodextrin concentration such that upon administration of the liposome to the subject, the liposome exhibits a uniform release profile of the hydrophobic lactone drug.

Among other factors, the present liposome may exhibit increased drug loading, prolonged drug retention, and prolonged drug release.

Other methods, features and advantages of the present invention will be or become apparent to one with skill in the art upon examination of the following detailed descriptions. It is intended that all such additional methods, features and advantages be included within this description, be within the scope of the present invention, and be protected by the accompanying claims.

### BRIEF DESCRIPTION OF THE DRAWINGS

FIG. 1 illustrates loading of DB-67 in the presence or absence of 0.1 M hydroxypropyl-β-cyclodextrin and varying intraliposomal pH.

FIG. 2 illustrates release of DB-67 from liposome encapsulated DB-67/hydroxypropyl-β-cyclodextrin complexes at an intravesicular pH of 4, with an extravesicular pH of 7.4.

FIG. 3 illustrates release of DB-67 from liposome encapsulated DB-67/hydroxypropyl-β-cyclodextrin complexes at pH 7.4

FIG. 4 illustrates drug retention of DB-67 in liposome encapsulated DB-67/hydroxypropyl-β-cyclodextrin (50 mM) complexes as a function of pH.

FIG. 5 illustrates in vivo release of nonliposomal DB-67 and release of DB-67 from liposomes prepared at high intravesicular pH with hydroxypropyl-β-cyclodextrin.

FIG. 6 illustrates tumor volume as a function of time during treatment of non-small cell lung cancer (H460) in mice with various dosages of nonliposomal DB-67.

FIG. 7 illustrates a dosing schedule and survival fraction during treatment of non-small cell lung cancer (H460) in mice with various dosages of nonliposomal DB-67.

### DETAILED DESCRIPTION

Before the present compositions and methods are described, it is to be understood that the invention is not limited to the particular methodologies, protocols, assays, and reagents described, as these may vary. It is also to be understood that the terminology used herein is intended to describe particular embodiments of the present invention, and is in no way intended to limit the scope of the present invention as set forth in the appended claims.

It must be noted that as used herein and in the appended claims, the singular forms “a,” “an,” and “the” include plural references unless the context clearly dictates otherwise.

Unless defined otherwise, all technical and scientific terms used herein have the same meanings as commonly understood by one of ordinary skill in the art to which this invention belongs. All publications cited herein are incorporated herein by reference in their entirety for the purpose of describing and disclosing the methodologies, reagents, and tools reported in the publications that might be used in connection with the invention. Nothing herein is to be construed as an admission that the invention is not entitled to antedate such disclosure by virtue of prior invention.

As used herein, the term “pharmaceutically effective amount” refers to an amount of an agent, reagent, compound, composition, or combination of reagents disclosed herein that, when administered to a subject, is sufficient to be effective against the disease state, including cancer.

The term “cancer” embraces a collection of malignancies with each cancer of each organ consisting of numerous subsets. Typically, at the time of cancer diagnosis, cancer consists in fact of multiple subpopulations of cells with diverse genetic, biochemical, immunologic, and biologic characteristics. Cancers may include, but are not limited to melanomas (e.g., cutaneous melanoma, metastatic melanomas, and intraocular melanomas), prostate cancer, lymphomas (e.g., cutaneous T-cell lymphoma, mycosis fungicides, Hodgkin's and non-Hodgkin's lymphomas, and primary central nervous system lymphomas), leukemias (e.g., pre-B cell acute lymphoblastic leukemia, chronic and acute lymphocytic leukemia, chronic and acute myelogenous leukemia, adult acute lymphoblastic leukemia, mature B-cell acute lymphoblastic leukemia, prolymphocytic leukemia, hairy cell leukemia, and T-cell chronic lymphocytic leukemia), and metastatic tumor. Cancer may be a solid tumor or a liquid tumor.

### Liposome

Disclosed herein are liposomes effective for delivering pharmaceutically effective amounts of hydrophobic lactone drugs.

Hydrophobic lactone drugs are present in different forms depending upon the pH of their environment. Hydrophobic lactone drugs contain a labile lactone moiety, which can undergo pH dependent reversible hydrolysis. Thus, the term “hydrophobic lactone drug” as used herein refers to a compound containing a labile lactone moiety that can be present in a form where the lactone ring is closed at low pH or a form where the lactone ring is open at high pH.

Camptothecins are exemplary hydrophobic lactone drugs and are a prominent class of chemotherapeutic agents that are cell cycle S-phase specific. The anti-cancer activity of camptothecins is primarily attributed to the intact lactone (Hertzberg et al., J. Med. Chem., 32(3): 715-720, 1989). In aqueous solution, camptothecins undergo a pH dependent lactone ring hydrolysis to form inactive carboxylate species (Fassberg et al., J. Pharm. Sci., 81(7): 676-684,1992). Typically, for camptothecins, the lactone ring-opened form will be inactive and the lactone ring-closed form will be an active form of the drug, although this is not required (i.e. both forms may be active or at least exhibit some activity).

Camptothecins are typically used to treat cancer, including malignant solid tumors. DB-67 is a camptothecin that has displayed excellent anti-cancer activity in cell culture and small animal studies (Bom et al., J. Med. Chem., 42(16): 3018-3022, 1999; Bom et al., J. Med. Chem., 43(21): 3970-3980, 2000; Pollack et al., Cancer Res., 59: 4898-4905, 1999) and is currently in Phase I clinical studies at the University of Kentucky Markey Cancer Center. While human data are not yet available for DB-67, animal data indicate that its lactone to total AUC after intravenous administration is >90%. As a result of this outstanding stability, DB-67 may have pharmacologic and pharmacokinetic advantages over the currently approved camptothecins and many currently in development. DB-67, along with gimatecan and karenitecin, represents a new generation of camptothecin analogs that exhibit good blood stability and enhanced lipophilicity and potency.

Exemplary camptothecins include, but are not limited to, camptothecin, silatecan 7-t-butyldimethylsilyl-10-hydroxycamptothecin (DB-67), 7-ethyl-10-hydroxy-20(S)-camptothecin (SN-38), topotecan, irinotecan, 9-nitro-camptothecin, lurtotecan, exatecan, gimatecan, and karenitecin.

DB-67 is an exemplary camptothecin. The forms of DB-67 illustrated below demonstrate the pH dependent reversible hydrolysis of hydrophobic lactone drugs that transforms the drugs from their lactone ring-closed form to their lactone ring-opened form.

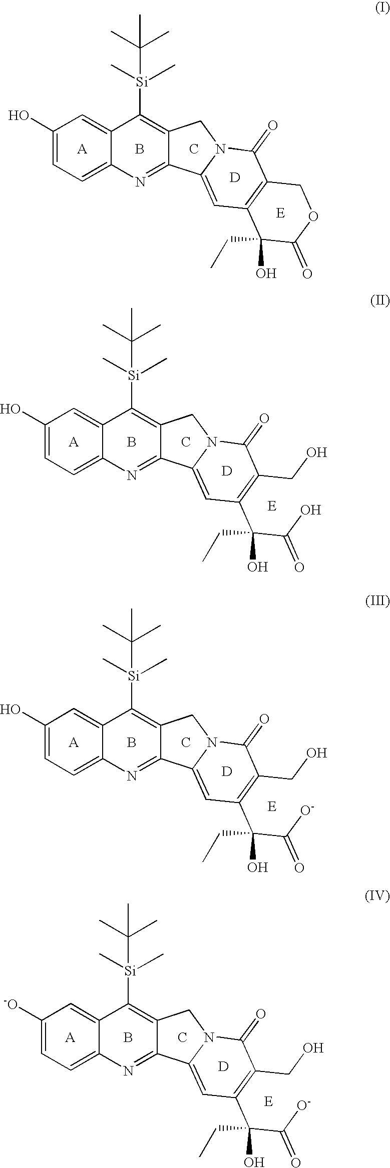

As shown above, DB-67, like other camptothecins, is subject to a pH dependent reversible hydrolysis of the α-hydroxy δ-lactone ring (E-ring) moiety to form DB-67 carboxylate anions. As a result, four different species of DB-67 exist depending upon pH. These four species are DB-67 lactone (I), DB-67 carboxylic acid (II), DB-67 carboxylate monoanion (III) and dianion (IV). DB-67 lactone (I) is the lactone ring-closed form of the drug and is membrane permeable.

Certain compounds of the camptothecin class possess an ionizable amine. These compounds are referred to herein as “ionizable amine-containing camptothecins.” Ionizable amine-containing camptothecins exist predominantly as cationic species at low pH (e.g. pH 2-7). Exemplary ionizable amine-containing camptothecins include lurotecan, topotecan, and irinotecan.

Other exemplary hydrophobic lactone drugs include, but are not limited to, statins, parthenolides, candimine, himbacine, narcotine, hydrastine, and homolycorine. It is well known in the art that the lactone moiety of statins, parthenolides, candimine, himbacine, narcotine, hydrastine, and homolycorine can undergo reversible hydrolysis.

Liposomal delivery is currently being investigated for various camptothecin analogues and several of these formulations are currently in preclinical or clinical trials (Emerson et al., Clin. Cancer Res., 6(7): 2903-2912, 2000; Colbern et al., Clin. Cancer Res., 4(12): 3077-3082, 1998; Tardi et al., Cancer Res., 60(13): 3389-3393, 2000; Messerer et al., Clin. Cancer Res., 10(19): 6638-6649, 2004; Pal et al., Anticancer Res., 25(1A): 331-341, 2005; Seiden et al., Gynecol. Oncol., 93(1): 229-232, 2004).

The challenge of delivering camptothecins and similar hydrophobic lactone drugs in liposomes lies in loading them in the liposomes, retaining them in the liposomes, and prolonging their release from the liposomes. Loading at therapeutic concentrations is complicated by their poor aqueous solubility. Prolonged retention is desired for liposome accumulation in the target tissue prior to drug release. Prolonged release is desired for less frequent drug dosing.

Efficient liposomal loading of camptothecins and similar hydrophobic lactone drugs is compromised by their poor aqueous solubility. To prepare liposomes by conventional methods such as hydration-extrusion or sonication, the drug must first be dissolved in an aqueous buffer. Alternatively, the drug is mixed with the lipid of interest in a suitable organic solvent and the solvent is evaporated to make a drug-lipid film. Then the film is hydrated with a polar solvent, such as water, to make liposomes. Thus, regardless of the method of preparation, an aqueous buffer has to be added at some stage of the formulation to make liposomes. Accordingly, liposomal loading of camptothecins and similar hydrophobic lactone drugs at therapeutically required concentration is thus impeded by their poor aqueous solubility.

In addition to loading challenges due to poor solubility, camptothecins and similar hydrophobic lactone drugs are poorly retained in liposomes. Prolonged drug retention in liposomes is often desired for tissue specific drug targeting. For example, in the case of cancer chemotherapy, a prolonged retention in liposomes is desired to allow enough time for liposomes to accumulate in tumor tissue. In such case, premature leakage of the encapsulated drug results in exposure of the healthy tissue to the drug, causing undesirable side effects.

Liposomes would appear to be ideal delivery systems for camptothecins and similar hydrophobic lactone drugs, especially if their release from the liposomes could be prolonged. Being relatively small and relatively lipophilic molecules, camptothecins exhibit large volumes of distribution and a narrow therapeutic index due to their accessibility and toxicity to normal tissues. However, long-circulating pegylated liposomes may reduce camptothecin distribution and toxicity in normal tissue. Long-circulating liposomes offer the possibility of prolonged drug release with less frequent dosing. Several studies have demonstrated the advantages of protracted camptothecin therapy (i.e., infusion regimens or multiple dosing over relatively frequent time intervals), but frequent dosing schedules are inconvenient to the patient and increase the cost of therapy. If camptothecin release from liposomes could be adequately prolonged, their activity could be extended allowing lower overall doses. Prolonged release needs to be addressed for similar hydrophobic lactone drugs as well.

The potential of using a high intraliposomal pH to maintain DB-67 in its membrane impermeable carboxylate monoanion (III) form was recently explored in order to develop prolonged release liposomal suspensions. V. Joguparthi, and B. D. Anderson. Liposomal delivery of hydrophobic weak acids: enhancement of drug retention using a high intraliposomal pH. J. Pharm. Sci. 97:433-454 (2008) and V. Joguparthi, T. X. Xiang, and B. D. Anderson. Liposome transport of hydrophobic drugs: gel phase lipid bilayer permeability and partitioning of the lactone form of a hydrophobic camptothecin, DB-76. J. Pharm. Sci. 97:400-420 (2008). However, a high intraliposomal pH could not be maintained under physiological conditions due to the rapid dissipation of the trans-membrane pH gradient by carbonate buffer (CO₂/H₂CO₃). V. Joguparthi, and B. D. Anderson. Liposomal delivery of hydrophobic weak acids: enhancement of drug retention using a high intraliposomal pH. J. Pharm. Sci. 97:433-454 (2008) and V. Joguparthi, S. Feng, and B. D. Anderson. Determination of intraliposomal pH and its effect on membrane partitioning and passive loading of a hydrophobic camptothecin, DB-67. Int. J. Pharm. 352:17-28 (2008). This inability to maintain a high intravesicular pH stimulated a search for alternative strategies to improve the retention of DB-67 and other similar hydrophobic lactone drugs.

Accordingly, the present invention provides a liposome which comprises a hydrophobic lactone drug and a cyclodextrin, and has an intraliposomal pH and a cyclodextrin concentration such that upon administration of the liposome to a subject, the liposome exhibits a uniform release profile of the hydrophobic lactone drug. The combination of intraliposomal pH and cyclodextrin can increase loading of the hydrophobic lactone drug in the liposome. The combination of intraliposomal pH and a cyclodextrin can also improve retention of the hydrophobic lactone drug in the liposome. The liposome does not exhibit biphasic, discontinuous release kinetics over the release profile. Moreover, release of the drug is typically prolonged relative to release from a liposome in which biphasic release kinetics occurs or which does not contain cyclodextrin.

In one embodiment, the liposome has an intraliposomal pH sufficiently high to maintain the hydrophobic lactone drug in its lactone ring-opened form. Thus, the intraliposomal pH at the time of administration can be any pH at which the hydrophobic lactone drug exists in its lactone ring-opened form. Such a lactone ring-opened form can include the opening of a lactone ring to form a carboxylate moiety.

While not wishing to be bound by any particular theory, it is believed that retention of the hydrophobic lactone drug in the liposome is promoted by the drug being present in a membrane impermeable lactone ring-opened form (e.g. DB-67 carboxylate anion (III)) and complexed with cyclodextrin at high pH. Surprisingly, conversion to the lactone ring-closed form (e.g. DB-67 lactone (I)) allows the drug to slowly permeate the liposome for its release. The release of the lactone ring-closed form is believed to be further prolonged due to the complexation of the membrane impermeable lactone ring-opened form with a cyclodextrin. In particular, at low pH (e.g. pH 4-7) the hydrophobic lactone drug exists predominantly as the lactone ring-closed form, which binds to the liposome membrane. The lactone ring-closed form that favors membrane association also favors membrane permeation. However, upon lactone ring opening at high pH, the fraction of drug bound to the membrane decreases while the fractions of complexed and free drug increase due to the relatively greater affinity of the lactone ring-opened form for the cyclodextrin compared to the membrane. Thus, the combination of intraliposomal cyclodextrin and a high intraliposomal pH appears to be responsible for the prolonged release of drug.

By adjusting the intraliposomal pH from higher to lower, with or without a concomitant change in the cyclodextrin concentration, a liposome with a quicker release profile can be produced. Hence, by appropriate adjustment of the intraliposomal pH and cyclodextrin concentration, a liposome can be generated with a release profile that is optimal for the particular patient and/or disease being treated.

The liposome of the present invention does not show a burst release that is typical of other drug/cyclodextrin complexes entrapped in liposomes. Unusual release kinetics characterized by an initial burst release followed by a second phase with slow or no drug release have been observed in several studies. D. G. Fatouros, K. Hatzidimitriou, and S. G. Antimisiaris. Liposomes encapsulating prednisolone and prednisolone—cyclodextrin complexes: comparison of membrane integrity and drug release. Eur. J. Pharm. Sci. 13:287-296 (2001). G. Piel, M. Piette, V. Barillaro, D. Castagne, B. Evrard, and L. Delattre. Betamethasone-in-cyclodextrin-in-liposome: the effect of cyclodextrins on encapsulation efficiency and release kinetics. Int. J. Pharm. 312:75-82 (2006). Thus, the liposomes are unexpectedly advantageous because they do not exhibit such biphasic release kinetics, but rather exhibit a uniform release profile. In fact, in one embodiment, the liposome exhibits first order release kinetics.

In one embodiment, the intraliposomal pH can be between about 6 and about 10. When the hydrophobic lactone drug is DB-67, the intraliposomal pH should be >7. This is due to the fact that, at pH<5, DB-67 is found primarily in its lactone ring-closed form. However, at pH>7, DB-67 is found primarily in its lactone ring-opened form. With DB-67, preferably the intraliposomal pH is between about 8 and about 10. With less hydrophobic camptothecins, preferably the intraliposomal pH is between about 6 and about 8. With parthenolides, the intraliposomal pH can be between about 6 and about 8.

In another embodiment, the hydrophobic lactone drug is in a lactone ring-closed form at an intraliposomal pH of 4.

As discussed above, the present liposome exploits the pH dependent reversible hydrolysis of the lactone moiety of the hydrophobic lactone drug to improve drug loading, retention, and release. Camptothecins are an exemplary class of hydrophobic lactone drug that can be used in the present liposome. Any camptothecin can be used. As discussed above, camptothecins are a prominent class of chemotherapeutic agents. Accordingly, when the present liposomes incorporate camptothecins including DB-67, they can be used to treat cancer, including malignant solid tumors.

Statins can be used in the present liposome and are another exemplary class of hydrophobic lactone drug. Exemplary statins include, but are not limited to, atorvastatin, cerivastatin, fluvastatin, lovastatin, mevastatin, pitavastatin, pravastatin, rosuvastatin, and simvastatin.

Statins are a prominent class of drugs that lower cholesterol in subjects with, or susceptible to, cardiovascular disease. Accordingly, when the present liposomes incorporate statins, they can be used to treat high cholesterol and/or treat cardiovascular disease.

Statins may also have utility in the treatment of cancer. See, for example, K. Hindler, C. S. Cleeland, E. Rivera, and C. D. Collard, The Role of Statins in Cancer Therapy, The Oncologist, Vol. 11, No. 3, 306-315, March 2006. Accordingly, when the present liposomes incorporate statins, they may also be used to treat cancer.

In one embodiment, the hydrophobic lactone drug is poorly soluble in water. In particular, in this embodiment, the hydrophobic lactone drug has an intrinsic solubility (i.e. the solubility of the lactone ring-closed form) of less than 1 mg/ml. Exemplary hydrophobic lactone drugs having such intrinsic solubility include, but are not limited to, camptothecins, statins, and parthenolides.

Any suitable cyclodextrin may be used to form the complex of cyclodextrin and hydrophobic lactone drug. Such cyclodextrins may include any of the α-cyclodextrins (six sugar ring molecules), β-cyclodextrins (seven sugar ring molecules), and γ-cyclodextrins (eight sugar ring molecules). Such cyclodextrins may further include cyclodextrins having five sugar ring molecules or greater than eight sugar ring molecules, such as cyclodextrins having up to 32 sugar ring molecules or an even greater number of sugar ring molecules, such as at least 150.

Natural cyclodextrins may be used to form the complex with the hydrophobic lactone drug. Exemplary natural cyclodextrins include, but are not limited to, α-, β-, and γ-cyclodextrins. Modified natural cyclodextrins may also be used to form the complex with the hydrophobic lactone drug. Exemplary modified natural cyclodextrins include, but are not limited to, glucosyl-α-cyclodextrin, glucosyl-β-cyclodextrin, glucosyl-γ-cyclodextrin, maltosyl-α-cyclodextrin, maltosyl-β-cyclodextrin, and maltosyl-γ-cyclodextrin.

In addition, chemically modified cyclodextrins may be used. Exemplary chemically modified cyclodextrins include, but are not limited to, methyl-α-cyclodextrin, methyl-β-cyclodextrin, methyl-γ-cyclodextrin, hydroxyethyl-α-cyclodextrin, hydroxyethyl-β-cyclodextrin, hydroxyethyl-γ-cyclodextrin, 2-hydroxypropyl-α-cyclodextrin, 2-hydroxypropyl-β-cyclodextrin, 2-hydroxypropyl-γ-cyclodextrin, carboxymethyl-α-cyclodextrin, carboxymethyl-β-cyclodextrin, carboxymethyl-γ-cyclodextrin, carboxyethyl-α-cyclodextrin, carboxyethyl-β-cyclodextrin, carboxyethyl-γ-cyclodextrin, sulfobutyl ether α-cyclodextrin, sulfobutyl ether β-cyclodextrin, and sulfobutyl ether γ-cyclodextrin. Additional chemically modified cyclodextrins which may be used include α-, β-, and γ-cyclodextrins modified with one or more substituents selected from among a sulfonic acid group, a sulfonic acid salt group, an ammonium group, a phosphoric acid group, a carboxyl group, a carboxylic acid salt group and a hydroxyl group, such as described in U.S. Pat. No. 5,241,059.

In one embodiment, the cyclodextrin is selected from the group consisting of β-cyclodextrin, analogs thereof, and derivatives thereof.

In another embodiment, the cyclodextrin is sulfobutyl ether-β-cyclodextrin (SBEβCD) or hydroxypropyl β-cyclodextrin (HPβCD). SBEβCD and HPβCD are approved for use in humans. Morever, HPβBD and SBEβCD do not appear to effect membrane stability. It is believed that some cyclodextrins affect membrane stability. For example, some cyclodextrins such as methyl-β-cyclodextrin can bind to lipids and alter their thermotropic properties. However, other cyclodextrins such as HPβCD and SBEβCD do not appear to interact with the bilayer.

In yet another embodiment, the total solute concentration (including the hydrophobic lactone drug, cyclodextrin, and any buffer) of the aqueous compartment in the liposome at the time of administration is not greater than 0.4 M. Any drug or excipients bound to the membrane or in undissolved solid form (such as entrapped precipitates and salts) are not considered toward the total concentration.

In a further embodiment, at the time of administration, the vesicles are unilamellar. Unilamellar vesicles constitute only a single lipid bilayer as opposed to multilamellar vesicles that constitute multiple lipid bilayers.

### Liposome Preparation

Procedures involved in preparing the present liposomes may include, for example, methods of preparing drug/cyclodextrin complex in solution, methods of preparing liposomes, methods of post-peglyation of liposomes, and methods of separating unentrapped drug/cyclodextrin complex from liposome entrapped drug/cyclodextrin complex. Exemplary procedures used to prepare the present liposomes are discussed below. Methods of preparing drug solution, cyclodextrin solution, and liposomes are well known in the art, as are liposomal separation methods. Accordingly, one skilled in the art could readily prepare the present liposomes from conventional techniques.

### Preparation of Drug/Cyclodextrin Complex in Solution

Generally, prior to making liposomes, a solution containing both drug and cyclodextrin is first prepared. To prepare a drug/cyclodextrin complex solution at a desired pH, the cyclodextrin and the drug can be dissolved in an aqueous buffer at the desired pH.

The pH of the buffer is chosen based on the drug candidate and is typically any pH at which the drug exists predominately in its lactone ring-opened form. For example, in the case of DB-67, the pH of the buffer is any pH>7. Preferably, this chosen pH is also the desired intraliposomal pH.

The buffer may contain anionic (e.g. carbonate, borate, phosphate) or cationic (e.g. Tris-HCl, ammonium hydroxide, TEA) acids and/or conjugate bases. The cyclodextrin chosen is preferably a neutral cyclodextrin such as hydroxypropyl-β-cyclodextrin. The cyclodextrin concentration can be varied to achieve a desired final release rate from the liposomes. Preferably, the concentration of the cyclodextrin in the liposomes is less than 0.3 M.

The drug/cyclodextrin complex in solution can be prepared by directly adding a weighed amount of drug to the cyclodextrin solution having a desired pH. Alternatively, a concentrate of the hydrophobic lactone drug in an organic solvent or weak base can be diluted into the cyclodextrin solution. In a preferred preparation method, a drug concentrate is first prepared in a basic solution (e.g. 0.1 N NaOH) and titrated into the cyclodextrin solution to obtain a desired concentration and pH. The final pH of the drug/cyclodextrin solution is any pH at which the chosen drug candidate predominately exists in its lactone ring-opened form.

In one embodiment, the drug/cyclodextrin complex is prepared by titration of drug solution with cyclodextrin prepared in aqueous buffers. Drug or drug/cyclodextrin solution is preferably prepared in aqueous solvents rather than organic solvent or water-organic mixtures.

### Preparation of Liposomes

The hydration-extrusion method, a method well known in the art, can be used to prepare the liposomes. Using the hydration-extrusion method, a freshly prepared drug/cyclodextrin complex in solution can be used to hydrate phospholipids of choice to prepare multilamellar vesicles. The multilamellar vesicles are then extruded through a membrane of desired pore size (typically 50-200 nm) to prepare unilamellar vesicles.

Alternatively, sonication, another method well known in the art, can be used to prepare unilamellar vesicles.

Preferably, the final liposome suspension comprises a mixture of phospholipids and may also include cholesterol.

A first phospholipid that can be used includes distearoylphosphatidyl choline (DSPC), dipalmitoylphosphatidyl choline (DPPC), diarachidonoyl phosphatidyl choline (DAPC), hydrogenated soy phosphatidyl choline (HSPC), dimyristoylphosphatidyl glycerol (DMPG), dioleoylphosphatidylglycerol (DOPG), dimyristoylphosphatidylcholine (DMPC), phosphatidyl choline (PC), and phosphatidyl ethanolamine (PE). This first phospholipid is usually the major component in the mixture of phospholipids and generally constitutes 70-95% of the mixture.

A second phospholipid that can be used includes distearoylphosphatidic acid (DSPA), hydrogenated soy phosphatidic acid (HSPC), dimyristoylphosphatidic acid (DMPA), phosphatidic acid (PA), etc. This second phospholipid is typically about 5-10% of the mixture. This second phospholipid is usually used to produce a surface charge on the liposomes to improve physical stability of the vesicles.

A third phospholipid that can be used is a pegylated phospholipid. This third phospholipid is typically about 5-10% of the mixture. Pegylation refers to the attachment of a polyethylene glycol segment to the phospholipid. This third phospholipid is usually pegylated phosphatidyl ethanolamine (PE) of any chain length. The pegylated phospholipid can be added either during preparation of unilamellar vesicles or added after preparation of unilamellar vesicles.

Another constituent in the liposomes can be cholesterol. Cholesterol can be used as an alternative to a pegylated phospholipid. Alternatively, cholesterol can be used in addition to a pegylated phospholipid.

All the phospholipid components in the liposomes preferably have the same chain length. For example, DSPC is preferably used with DSPA and pegylated DSPE.

During the hydration procedure, it is preferable to use only the major lipid component (for example: DSPC), which may be in combination with cholesterol and a physical stabilizer (DSPA) to prepare liposomes. Other components such as a pegylated phospholipid are preferably added after preparation of the unilamellar vesicles. When liposomes are pegylated after preparation, this process is usually referred to as post-pegylation.

### Post-Pegylation of Liposomes

If liposomes are to be post-pegylated, a concentrated micelle solution of the pegylated phospholipid is first prepared in the same buffer used to prepare the unilamellar vesicles. A small amount of this stock solution is then added to the vesicle suspension immediately following preparation of unilamellar vesicles and incubated for at least 10 minutes at 60° C. Alternatively, the pegylated phospholipid can be added after the vesicles have been allowed to cool at room temperature and following addition the liposomes are incubated for at least 20 minutes at room temperature. If the half-life of drug release is greater than 10 hours, then generally liposomes are post-pegylated, preferably at 60° C., after removal of unentrapped drug/cyclodextrin complex.

Separation of Unentrapped Drug/Cyclodextrin Complex from Liposome Entrapped Drug/Cyclodextrin Complex

The liposomes are separated from unentrapped drug/cyclodextrin complex, for example, at the time of administration, by any commonly used separation method. Alternatively, the liposomes can be separated from unentrapped drug/cyclodextrin complex a few hours after preparation and then frozen or lyophilized until use. Separation methods which can be used include gel filtration, ultrafiltration, centrifugation, or dialysis. During the separation of liposomally entrapped from unentrapped drug, the buffer type and the total solute concentration of the exchange buffer are similar to those in the entrapped aqueous compartment. The solute concentration for cyclodextrin and drug in the exchange buffer are substituted by the liposome membrane impermeable solutes such as sucrose or sodium chloride.

### Method of Administration

Further disclosed herein is a method of administering a hydrophobic lactone drug to a subject in need thereof by use of the present liposomes. Such method includes administering a liposome to a subject in need. The liposome comprises the hydrophobic lactone drug and a cyclodextrin and has an intraliposomal pH and a cyclodextrin concentration such that upon administration of the liposome to the subject, the liposome exhibits a uniform release profile of the hydrophobic lactone drug.

The method of administration may deliver pharmaceutically effective amounts of the hydrophobic lactone drug to a target tissue or to the bloodstream.

To deliver pharmaceutically effective amounts of the hydrophobic lactone drug to a target tissue, the liposomes should generally exhibit several characteristics. Namely, the concentration of the drug encapsulated in the liposomes should be sufficient to enable administration of pharmaceutically effective doses in vivo. After administration, the drug should be sufficiently retained while the liposomes are circulating in the bloodstream to prevent its leakage from the liposomes before the liposomes have collected in the target tissue, such as a tumor. Once the liposomes have collected in the target tissue, the rate of drug release should not be so slow that the tissue levels of the drug fail to reach adequate concentrations for effective treatment (e.g. effective tumor cell killing/growth inhibition). The active form of the drug should be delivered to the target tissue.

Such method of administration can be used to treat cancer or, alternatively, to treat high cholesterol. When the hydrophobic lactone drug of the liposome is a camptothecin or a statin, the method of administration may be used to treat cancer in a subject. The cancer may be in the form of a solid tumor. Similarly, when the hydrophobic lactone drug of the liposome is a statin, the method of administration may be used to treat high cholesterol.

The subject in need can be any mammalian species including, but not limited to, human, monkey, cow, sheep, pig, goat, horse, mouse, rat, dog, cat, rabbit, guinea pig, hamster, and horse. Preferably the subject in need is human.

The present liposomes can be delivered directly or in pharmaceutical compositions along with suitable carriers or excipients, as is well known in the art. For example, a pharmaceutical composition may include a conventional additive, such as a stabilizer, buffer, salt, preservative, filler and the like, as known to those skilled in the art. Exemplary buffers include phosphates, carbonates, citrates, and the like. Exemplary preservatives include EDTA, EGTA, BHA, BHT and the like.

A pharmaceutically effective amount of the drug can readily be determined by routine experimentation, as can the most effective and convenient route of administration and the most appropriate formulation. Various formulations and drug delivery systems are available in the art. See, e.g., Gennaro, A. R., ed. (1995) Remington's Pharmaceutical Sciences.

Suitable routes of administration may, for example, include oral, topical, transdermal, local by inhalation, rectal, transmucosal, nasal, or intestinal administration and parenteral delivery, including intramuscular, subcutaneous, intramedullary injections, as well as intrathecal, direct intraventricular, intravenous, intraperitoneal, intranasal, or intraocular injections. In addition, the formulations may be administered sublingually or via an aerosol or spray, including a sublingual tablet or a sublingual spray. The formulations may be administered in a local rather than a systemic manner. For example, a suitable formulation can be delivered via injection or in a targeted drug delivery system, such as a depot or sustained release formulation. Other uses, depending on the particular properties of the preparation, may be envisioned by those skilled in the art.

The mode of administration of the liposomes and pharmaceutical formulations thereof may determine the sites and cells in the subject to which the hydrophobic lactone drug will be delivered. The liposomes of the present invention can be administered alone but will generally be administered in admixture with a pharmaceutical carrier selected with regard to the intended route of administration and standard pharmaceutical practice.

As discussed above, the preparations may be injected parenterally, for example, intravenously. Preferably, the route of administration is intravenous. For parenteral administration, they can be used, for example, in the form of a sterile aqueous solution which may contain other solutes, for example, enough salts or glucose to make the solution isotonic. They may also be employed for peritoneal lavage or intrathecal administration via injection. They may also be administered subcutaneously.

For the oral mode of administration, the liposomes and pharmaceutical formulations thereof can be used in the form of tablets, capsules, losenges, troches, powders, syrups, elixirs, aqueous solutions and suspensions, and the like. In the case of tablets, carriers which can be used include lactose, sodium citrate and salts of phosphoric acid. Various disintegrants such as starch, and lubricating agents, such as magnesium stearate, sodium lauryl sulfate and talc, are commonly used in tablets. For oral administration in capsule form, useful diluents are lactose and high molecular weight polyethylene glycols. When aqueous suspensions are required for oral use, the active ingredient is combined with emulsifying and suspending agents. If desired, certain sweetening and/or flavoring agents can be added.

For the topical mode of administration, the liposomes and pharmaceutical formulations thereof may be incorporated into dosage forms such as gels, oils, emulsions, and the like. Such preparations may be administered by direct application as a cream, paste, ointment, gel, lotion or the like.

The pharmaceutical formulations may be manufactured by any of the methods well known in the art, such as by conventional mixing, dissolving granulating, dragee-making, levigating, emulsifying, encapsulating, entrapping, or lyphophilizing processes. As noted above, the formulations can include one or more physiologically acceptable carriers such as excipients and auxiliaries that facilitate processing of active molecules into preparations for pharmaceutical use.

Proper formulation is dependent upon the route of administration chosen. For injection, for example, the composition may be formulated in aqueous solutions, preferably in physiologically compatible buffers such as Hanks' solution, Ringer's solution, or physiological saline buffer. For transmucosal or nasal administration, penetrants appropriate to the barrier to be permeated are used in the formulation. Such penetrants are generally known in the art.

Compositions formulated for parenteral administration by injection, e.g., by bolus injection or continuous infusion can be presented in unit dosage form, e.g., in ampoules or in multi-dose containers, with an added preservative.

For any composition used in the present methods of administration, a pharmaceutically effective dose can be estimated initially using a variety of techniques well known in the art. For example, in a cell culture assay, a dose can be formulated in animal models to achieve a circulating concentration range that includes the IC₅₀ as determined in cell culture. Dosage ranges appropriate for human subjects can be determined, for example, using data obtained from cell culture assays and other animal studies.

A pharmaceutically effective dose of an agent refers to that amount of the agent that results in amelioration of symptoms or a prolongation of survival in a subject. Toxicity and therapeutic efficacy of such molecules can be determined by standard pharmaceutical procedures in cell culture or experimental animals, e.g., by determining the LD₅₀ (the dose lethal to 50% of the population) and the ED₅₀ (the dose pharmaceutically effective in 50% of the population). The dose ratio of toxic to therapeutic effects is the therapeutic index, which can be expressed as the ratio LD₅₀/ED₅₀. Agents that exhibit high therapeutic indices are preferred.

Dosages preferably fall within a range of circulating concentrations that includes the ED₅₀ with little or no toxicity. Dosages may vary within this range depending upon the dosage form employed and the route of administration utilized. The exact formulation, route of administration, and dosage should be chosen, according to methods known in the art, in view of the specifics of a subject's condition.

The amount of liposomal formulation administered will, or course, be dependent on a variety of factors, including the sex, age, and weight of the subject being treated, the severity of the affliction, the manner of administration, and the judgment of the prescribing physician.

The present compositions may, if desired, be presented in a pack or dispenser device containing one or more unit dosage forms containing the active ingredient. Such a pack or device may, for example, comprise metal or plastic foil, such as a blister pack. The pack or dispenser device may be accompanied by instructions for administration. Compositions comprising a composition of the invention formulated in a compatible pharmaceutical carrier may also be prepared, placed in an appropriate container, and labeled for treatment of an indicated condition.

These and other embodiments will readily occur to those of ordinary skill in the art in view of the disclosure herein, and are specifically contemplated.

### EXAMPLES

The invention is further understood by reference to the following examples, which are intended to be purely exemplary of the invention. The present invention is not limited in scope by the exemplified embodiments, which are intended as illustrations of single aspects of the invention only. Any methods that are functionally equivalent are within the scope of the invention. Various modifications of the invention in addition to those described herein will become apparent to those skilled in the art from the foregoing description. Such modifications fall within the scope of the appended claims.

### Example 1

### Liposome Preparation

A 30 mg/ml solution of DB-67 was prepared in 0.1 N NaOH solution and filtered through a 0.2 μm syringe filter. This drug solution was added to a 0.1 M hydroxypropyl-β-cyclodextrin solution in pH 7.4 phosphate buffer such that the drug concentration in the final suspension was 1.5 mg/ml. The drug/cyclodextrin solution was used to hydrate phospholipids (DSPC, 40 mg/ml) with vigorous shaking at 60° C. to form a suspension of multilamellar vesicles. The suspension was extruded through a high-pressure extruder to form unilamellar vesicles. A 100 mg/ml micelle solution of m-PEG DSPE was prepared in 1 ml of pH 7.4 phosphate buffer at 60° C. A small amount of this micellar solution was added to the unilamellar vesicles following extrusion and incubated at 60° C. for 5 min. The vesicles were then cooled at room temperature and stored below 5° C. until use. Prior to use, liposomes were separated from unentrapped drug by passing through a gel filtration column which was pre-equilibrated with pH 7.4 “carbonated” phosphate buffered saline (PBS). “Carbonated” PBS was prepared at physiological concentration of carbonate, phosphate and sodium chloride such that the total solute concentration of the buffer was isoosmotic with that of whole blood. The liposomes collected from gel filtration were immediately transferred into a dialysis tube and dialyzed at 37° C. against 1 liter of “carbonated” PBS. 100 μl samples were taken from inside the dialysis tube at various time intervals and diluted into 900 μl of ice-cold solution of methanol and acetonitrile (2:1, v/v) at −25° C. Following dilution samples were stored at −25° C. until HPLC analysis.

### Example 2

### Liposome Preparation

A 20 mg/ml solution of DB-67 was prepared in 0.1N NaOH and filtered through a 0.2 μm syringe filter. This drug solution was added to a 0.01 M sulfobutyl ether-β-cyclodextrin solution in pH 9 borate buffer such that the drug concentration in the final suspension was 1 mg/ml. The drug/cyclodextrin solution was used to hydrate phospholipids (DSPC +5 mol % m-PEG DSPE) with vigorous shaking at 60° C. to form a suspension of multilamellar vesicles (40 mg/ml lipid). The suspension was extruded through a high-pressure extruder to form unilamellar vesicles. The vesicles were then cooled at room temperature and stored below 5° C. until use. Prior to use, liposomes were separated from unentrapped drug by passing through a gel filtration column which was equilibrated with pH 7.4 “carbonated” phosphate buffer. 100 μl of the liposomes collected from gel filtration were immediately added to 4 ml of plasma and incubated at 37° C. 50 μl of plasma was taken at various times and diluted into 150 μl of ice-cold solution of methanol and acetonitrile (2:1, v/v) and centrifuged at −9° C. for 3 minutes. The supernatant was collected and stored at −25° C. until HPLC analysis of DB-67.

### Example 3

### Liposome Preparation

The unilamellar vesicles are prepared as in Examples 1 and 2 but the buffer used is pH 9 glycine.

### Example 4

### Liposome Preparation

The unilamellar vesicles are prepared as in Examples 1 and 2 but the buffer used is pH 9.5 carbonate.

### Example 5

### Liposome Preparation

The unilamellar vesicles are prepared as in Examples 1 and 2 but the buffer used is pH 9.5 Tris-HCl.

### Example 6

### Liposome Preparation

The unilamellar vesicles are prepared as in Examples 1 and 2 but the buffer used is pH 9.5 ammonium hydroxide.

### Example 7

### Liposome Preparation

The unilamellar vesicles are prepared as in Examples 1 and 2 but the drug/cyclodextrin solution is prepared in deionized water without the use of any buffer species.

### Example 8

### Liposome Preparation

The multilamellar vesicles are formed as in Examples 1 through 7 but unilamellar vesicles are formed by sonication rather extrusion.

### Example 9

### Liposome Preparation

The vesicles are prepared with any of the buffers or methods of liposome preparation used in Examples 1 through 8 but the drug candidate is any hydrophobic lactone ring containing compound.

### Example 10

### Liposome Preparation

The vesicles are prepared with any of the buffers or methods of liposome preparation or choice of drug candidate used in Examples 1 through 9, but the cyclodextrin chosen is any natural or synthetic cyclodextrin.

### Example 11

### Liposome Preparation

The vesicles are prepared with any of the buffers or methods of liposome preparation or choice of drug candidate and cyclodextrin used in Examples 1 through 10 but during the separation of entrapped from unentrapped drug, the extraliposomal buffer is exchanged for pH 4 citrate with the intraliposomal pH being the same as that used in liposome preparation.

### Example 12

### Liposome Preparation

The vesicles are prepared with any of the buffers, methods of preparation or choice of drug candidate and cyclodextrin used in Examples 1 through 10 but during the separation of entrapped from unentrapped drug, the extraliposomal buffer is exchanged for a desired concentration of NaCl solution with the intraliposomal pH being the same as that used in liposome preparation.

### Example 13

### Liposome Preparation

The vesicles are prepared with any of the buffers, methods of preparation or choice of drug candidate and cyclodextrin used in Examples 1 through 10 but during the separation of entrapped from unentrapped drug, the extraliposomal buffer is exchanged for a desired concentration of sucrose solution with the intraliposomal pH being the same as that used in liposome preparation.

### Example 14

### Liposome Preparation

The vesicles are prepared with any of the buffers, methods of preparation or choice of drug candidate and cyclodextrin used in Examples 1 through 10 but the phospholipids used are 90% DSPC and 10% m-PEG DSPE.

### Example 15

### Liposome Preparation

The vesicles are prepared with any of the buffers, methods of preparation or choice of drug candidate and cyclodextrin used in Examples 1 through 10 but the phospholipids used are 95% HSPC and 5% pegylated PE.

### Example 16

### Liposome Preparation

The vesicles are prepared with any of the buffers, methods of preparation or choice of drug candidate and cyclodextrin used in Examples 1 through 10 but the phospholipids used are 90% DSPC, 5% DSPA and 5% pegylated PE.

### Example 17

### Liposome Preparation

The vesicles are prepared with any of the buffers, methods of preparation or choice of drug candidate, cyclodextrin and phospholipids chosen in Examples 1 through 16, but the method of separation of unentrapped from entrapped drug/cyclodextrin complex is ultrafiltration.

### Example 18

### Liposome Preparation

The vesicles are prepared with any of the buffers, methods of preparation or choice of drug candidate, cyclodextrin and phospholipids chosen in Examples 1 through 16, but the method of separation of unentrapped from entrapped drug/cyclodextrin complex is equilibrium dialysis.

### Example 19

### Drug Loading

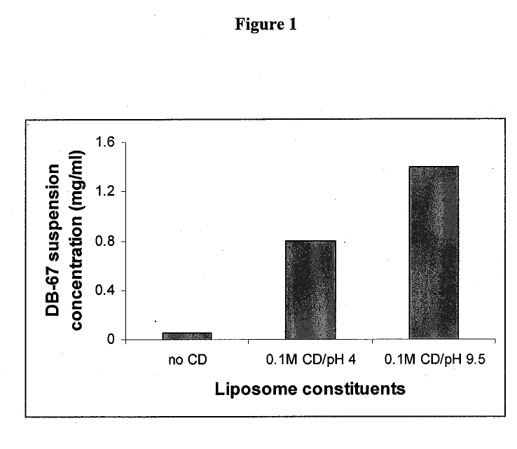

DB-67/hydroxypropyl-β-cyclodextrin complexes were entrapped in liposomes by a passive encapsulation technique at varying intraliposomal pH. The use of a high intraliposomal pH in combination with hydroxypropyl-β-cyclodextrin improves drug loading into vesicles (FIG. 1). In the absence of cyclodextrin, only 0.05 mg/ml suspensions of DB-67 (all drug is entrapped) were feasible but when the liposomes (at the same lipid concentration) were prepared at pH 4 using 0.1 M hydroxypropyl-β-cyclodextrin and DB-67 lactone, a 0.8 mg/ml suspension could be prepared. When the intraliposomal pH was increased to 9.5, the loading was further improved so that a 1.4 mg/ml liposome suspension could be prepared. Thus, the use of cyclodextrin in combination with high pH increases the amount of drug loaded into liposomes due to the improvement in solubility of DB-67 as a function of both pH and cyclodextrin.

### Example 20

### Uniform Release Profile and Drug Retention

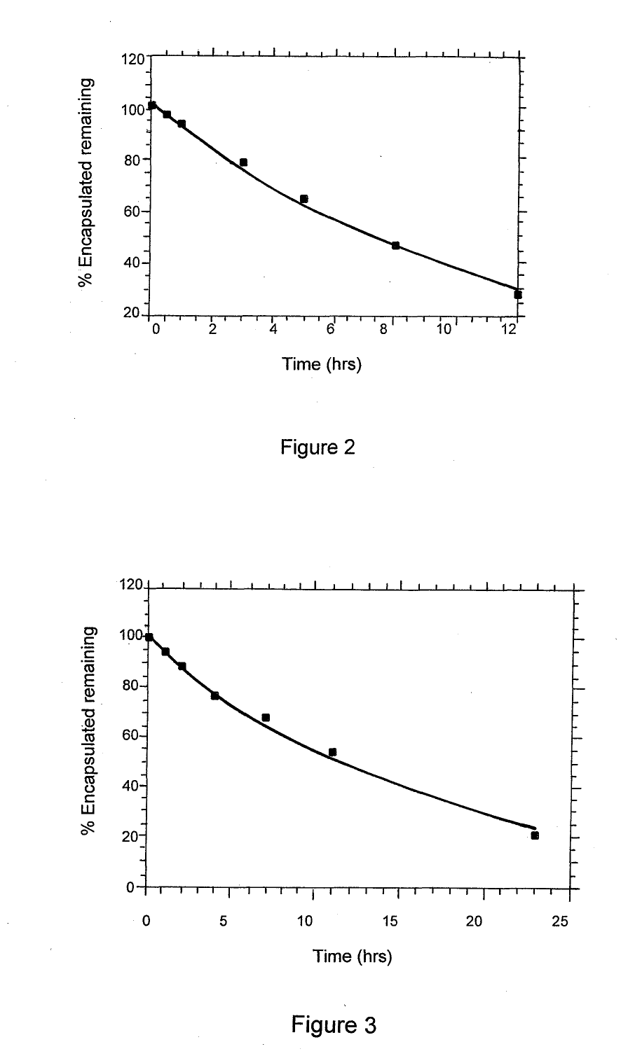

The ability to have a uniform drug release from liposomes entrapped with drug/cyclodextrin complexes without any burst release is typically observed with these systems. FIG. 2 shows the uniform release profile of DB-67 from liposomes prepared at pH 4 using a citrate buffer and 0.1 M hydroxypropyl-β-cyclodextrin. FIG. 3 shows the uniform release of drug from liposomes prepared at a pH of 7.4 using citrate buffer and 0.1 M hydroxypropyl-β-cyclodextrin. Thus, regardless of the pH used, these liposome systems display a uniform release profile in contrast with that in the existing literature.

Also demonstrated is the programmable prolongation of liposome retention of hydrophobic camptothecins by use of a high intraliposomal pH in the presence of cyclodextrin. Table 1 shows the liposome release half-life observed in the presence or absence of cyclodextrin using the same drug and lipid concentration but at varying pH and cyclodextrin concentration. The half-life for release can be increased up to more than 50 hours by use of a high formulation pH in the presence of hydroxypropyl-β-cyclodextrin. This half-life is significantly longer than those previously reported with camptothecins such as DB-67 and SN-38 and significantly greater than a liposomal formulation of DB-67 lactone that contains no cyclodextrin.

TABLE 1
Half-lives for liposome release of DB-67 in the presence or absence
of hydroxypropyl-β-cyclodextrin (HPβCD) at varying pH

| Formulation Conditions | Release half-life (hrs) |
|---|---|
| pH 4 | 3 |
| 0.1 M HPβCD/pH 4 | 6 |
| pH 7.4 | 3.5 |
| 0.1 M HPβCD/pH 7.4 | 12 |
| pH 9.85 | 12 |
| 0.05 M HPβCD/pH 9.85 | 63 |

### Example 21

### Drug Retention

Hydroxypropyl-β-cyclodextrin (HPβCD, degree of substitution=2.94, MW=1305.5) was obtained from American Maize-Products Company (Hammond, Ind.). DB-67 (7-t-butyl-dimethylsilyl-10-hydroxycamptothecin) was obtained from Novartis Pharmaceuticals Corporation (East Hanover, N.J., USA). Phospholipids (1,2-distearoyl-sn-glycero-3-phosphatidylcholine (DSPC) and 1,2-distearoyl-sn-glycero-3-phosphoethanolamine-N-[polyethylene glycol 2000] [m-PEG DSPE, MW=2,806)) were purchased as powders from Avanti Polar Lipids (Alabaster, Ala., USA). Sephadex® G-25M pre-packed size exclusion columns were purchased from GE Healthcare Bio-sciences Corporation (Piscataway, N.J.). Dialysis tubes (Float-A-Lyzer®, MWCO: 100,000) were obtained from Spectrum Laboratories (Rancho Dominguez, Calif., USA). All other reagents and HPLC solvents were obtained from Fischer Scientific (Florence, Ky., USA).

HPβCD solutions were prepared by adding a weighted amount of HPβCD to 2 mL of buffer (pH 3.5-9.5). Solutions were prepared at varying HPβCD concentrations (10-50 mM) at low pH (3.5-4) while solutions at pH>4 contained 50 mM HPβCD. An aliquot of stock solution of DB-67 in DMSO (1 mM) was added to the HPβCD solutions to provide DB-67 concentrations of 1-10 μM for use in preparing vesicles containing both HPβCD and DB-67. The final osmolality of each of the above solutions was adjusted to 300 mOsm with NaCl. Two milliliter aliquots of each HPβCD solution (with DB-67) were added to test tubes containing a 100 mg film of DSPC (prepared as described in V. Joguparthi, T. X. Xiang, and B. D. Anderson. Liposome transport of hydrophobic drugs: gel phase lipid bilayer permeability and partitioning of the lactone form of a hydrophobic camptothecin, DB-67. J. Pharm. Sci. 97: 400-420 (2008)). Unilamellar vesicle suspensions containing 50 mg DSPC/mL (diameter=200 nm) were prepared by the hydration-extrusion procedure (explained in V. Joguparthi, T. X. Xiang, and B. D. Anderson. Liposome transport of hydrophobic drugs: gel phase lipid bilayer permeability and partitioning of the lactone form of a hydrophobic camptothecin, DB-67. J. Pharm. Sci. 97: 400-420 (2008)) at 60° C. The final osmolalities of the extravesicular solutions in all vesicle preparations were adjusted to match the osmolality of the entrapped solution.

All vesicle suspensions containing HPβCD were pegylated following their preparation by first storing them at room temperature for 30 min, then transferring them into a dialysis tube to remove the unentrapped HPβCD by dialyzing against 1 L of buffer having a pH and osmolality matching that of the entrapped solution at 37° C. for 6 h. (The removal of unentrapped HPβCD by the dialysis method was validated in separate experiments using blank vesicles spiked with HPβCD). Following removal of the unentrapped HPβCD, the vesicle suspensions were equilibrated at 60° C. and pegylated by addition of stock solution of m-PEG DSPE (100 mg/mL (prepared in corresponding buffer)) to obtain vesicles pegylated on the outer lipid monolayer (5 mol %). Following addition of m-PEG DSPE, vesicles were stored at 60° C. for 1 h and for 2 h at room temperature.

The pH of all the buffers and vesicles was monitored at each step of the formulation procedure and the pH of the corresponding buffers was adjusted (with 0.1 N HCl or NaOH) as required to the pH of the vesicles. The particle size of the vesicles was measured by dynamic light scattering (DLS) using a Malvern Zetasizer-3000 (Malvern Instruments Ltd, Malvern, UK) at each step of the formulation procedure. DLS was also employed as described in V. Joguparthi, T. X. Xiang, and B. D. Anderson. Liposome transport of hydrophobic drugs: gel phase lipid bilayer permeability and partitioning of the lactone form of a hydrophobic camptothecin, DB-67. J. Pharm. Sci. 97: 400-420 (2008) to validate the Sephadext® column separation of liposome entrapped versus free drug in the presence of HPβCD. The osmolality of drug solutions and vesicles was measured by the freezing point depression method (Model 110 Osmometer, Fiske Associates, Norwood, Mass., USA). The osmolality of all the buffers used in these experiments was adjusted with NaCl to the internal osmolality of the vesicles to ensure that there were no osmolality gradients during the permeability studies.

The release of DB-67 from the vesicles as function of pH 4-8.5 was monitored by a dynamic dialysis method discussed in V. Joguparthi, T. X. Xiang, and B. D. Anderson. Liposome transport of hydrophobic drugs: gel phase lipid bilayer permeability and partitioning of the lactone form of a hydrophobic camptothecin, DB-67. J. Pharm. Sci. 97: 400-420 (2008). Liposomes were separated from the unentrapped drug and HPβCD by size exclusion chromatography on Sephadex® columns. At each pH, 0.1 mL of the liposome suspension was loaded onto a Sephadex® column (pre-conditioned with 50 mL of corresponding buffer) and eluted with 5 mL of buffer. The eluent liposome suspension (5 mL) was collected, immediately transferred to a dialysis tube, and dialyzed against 1 L of the same buffer at 37° C. At various times, 100 μL of liposome suspension was withdrawn from the dialysis tube and diluted into 900 μL of a mixture of cold (−25° C.) methanol/acetonitrile (2:1 (v/v)) to quench the carboxylate/lactone interconversion reaction. The pipette tip used for sampling was washed in the same quenching solution to transfer any adsorbed drug. The quenched samples were stored at −25° C. prior to their analysis for DB-67 lactone and carboxylate concentration by HPLC. Entrapped HPβCD concentration was determined after 1 mL of the original undiluted lipsome suspension (pH 3.5-8.5) was dialyzed for 2 h at 37° C. against 1 L of the corresponding buffer to remove any unentrapped cyclodextrin that was not completely removed during the dialysis step prior to pegylation. Following dialysis, samples were withdrawn from the dialysis tube, diluted into methanol, and stored at −25° C. until HPLC analysis.

The effect of intravesicular cyclodextrin (50 mM) on drug retention under physiological conditions (pH 7.4, 296 mOsm) was investigated by studying drug release from vesicles prepared from pH 9.5 buffer in pH 7.4 carbonated phosphate buffered saline (C-PBS) as described in V. Joguparthi and B. D. Anderson. Liposomal delivery of hydrophobic weak acids: enhancement of drug retention using a high intraliposomal pH. J. Pharm. Sci. 97: 433-454 (2008). In these studies, the extravesicular buffer was exchanged for C-PBS buffer by size exclusion similar to the permeability studies described earlier and the vesicles were dialyzed against 1 L of C-PBS buffer while taking samples at various time intervals. The sampling and processing conditions were the same as in the pH-permeability studies described earlier.

Drug (lactone or carboxylate) analyses utilized an isocratic HPLC method with fluorescence detection. Standards for DB-67 lactone and carboxylate were prepared in methanol and 10 mM carbonate buffer (pH 10.5), respectively. The solvents, columns, and chromatographic system employed in the analyses and the relevant method validations have been described in V. Joguparthi, T. X. Xiang, and B. D. Anderson. Liposome transport of hydrophobic drugs: gel phase lipid bilayer permeability and partitioning of the lactone form of a hydrophobic camptothecin, DB-67. J. Pharm. Sci. 97: 400-420 (2008).

HPβCD was analyzed by gradient HPLC with evaporative light scattering detection (ELSD, Sedere Inc., Lawrenceville, N.J., USA) using a Metasil® AQ (Metachem Technologies, Lake Forest, Calif., USA) C-18 column (120 Å, 250×46 mm) with a linear gradient starting at 100% methanol, changing to 50% methanol: 50% acetonitrile (v/v) in 5 min, and 100% acetonitrile in 10 min. The gradient was changed back to 100% methanol in 15 min and total run time was 20 min at a flow rate of 1 mL/min. The sample injection volume was 10 μL. The sample compartment and column holder were at ambient temperature. ELSD conditions included a gain of 8, temperature of 50° C. and pressure of 2.6 lb. Standards for HPβCD (100-500 μM) were prepared in methanol and all experimental samples were diluted to this concentration range in methanol for analysis. The detector response factor was calculated using a log concentration versus log peak area calibration curve. The retention time for HPβCD was approximately 5.5 min. The limit of quantitation was 10 μM.

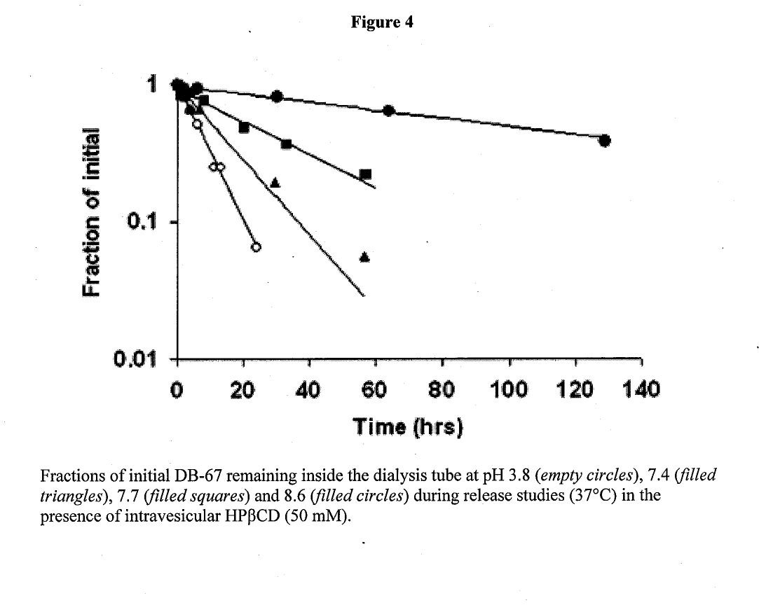

The effect of varying intravesicular pH on the apparent permeability was probed only at a single (50 mM) cyclodextrin concentration. The representative apparent release profiles for loss of DB-67 as a function of pH from inside the dialysis tube in the presence of 50 mM intravesicular cyclodextrin are shown in FIG. 4. As depicted in FIG. 4, increasing pH while holding the cyclodextrin concentration constant at 50 mM increases drug retention.

### Example 22

### Animal Pharmacokinetic Studies

Phospholipids 1,2-distearoyl-sn-glycero-3-phosphatidylcholine (DSPC, >99% purity) and 1,2-distearoyl-sn-glycero-3-phosphoethanolamine-N-[methoxy-(polyethyleneglycol)-2000] (m-PEG DSPE, MW=2806, >99% purity) were purchased as powders from Avanti Polar Lipids (Alabaster, Ala.). DB-67 was obtained from the Novartis Pharmaceuticals Corp. (East Hanover, N.J.). Blank plasma used in preparation of calibrators and quality control solutions was obtained from Abacell Corp. (San Mateo, Calif.). Consumables were treated with AquaSil™ siliconizing reagent (Pierce, Rockford, Ill.). Silconized pipet tips were obtained from Cole-Palmer and amber siliconized microcentrifuge tubes were obtained from Crystalgen Inc. (Plainview, N.Y.). Hydroxypropyl-β-cyclodextrin (HPβCD, degree of substitution=2.94, MW=1305.5) was obtained from American Maize-Products Company (Hammond, Ind.). Heparin (heparin sodium 1000 IU) was obtained from Baxter (Deer Field, Ill.). Dialysis tubes (Float-A-Lyzer®, MWCO: 100,000) and pre-cut dialysis membrane discs (MWCO: 12000-14000) were purchased from Spectrum Laboratories (Rancho Dominguez, CA). Sephadex® G-25 M pre-packed size exclusion columns were obtained from GE Healthcare Bio-sciences Corp. (Piscataway, N.J.). All other reagents were purchased from Fischer Scientific (Florence, Ky.) and HPLC grade solvents were obtained from VWR Scientific (Muskegon, Mich.).

A film of the desired lipids, DSPC and m-PEG-DSPE (95:5 mol %) was prepared by dissolving weighed amounts of lipids in chloroform, distributing into glass test tubes at 120 mg of total lipid per tube, evaporating chloroform under N₂ and drying in vacuo at 40° C. overnight. The lipid films were stored at 5° C. until use. These lipid films were employed in preparation of all liposomes employed in these studies.

Blank liposomes were prepared according to the following procedure. Two mL of 85 mM Na acetate buffer solution (pH 4, osmolality adjusted to 300 mOsm with NaCl) was added to a test tube containing 120 mg lipid film and blank unilamellar vesicles (DSPC: m-PEG DSPE 95:5 mol %) were prepared at 60 mg/mL (total lipid concentration) by the hydration-extrustion procedure (explained in V. Joguparthi, T. X. Xiang, and B. D. Anderson. Liposome transport of hydrophobic drugs: gel phase lipid bilayer permeability and partitioning of the lactone form of a hydrophobic camptothecin, DB-67. J. Pharm. Sci. 97: 400-420 (2008)) at 60° C. Following preparation, vesicles were allowed to cool down at room temperature for 2 h and were stored at 5° C. until use in animal injections. On the day of pharmokinetic studies, blank vesicles were warmed up at 37° C. and vesicles were spiked with DB-67 using a stock solution (100 mg/mL) of DB-67 in DMSO to obtain a 1 mg/mL lipsome suspension of DB-67. The spiked vesicles were immediately injected into animals at the desired dose (see dosing section).

Liposomes were prepared with high intraliposomal pH in the presence of cyclodextrin according to the following procedure. A 20 mM DB-67 solution was prepared in 70 mM Na carbonate buffer (pH 9.5) and 2 mL of this solution was supplemented (by adding a pre-weighed amount) with HPβCD to obtain a drug solution with 50 mM HPβCD. This solution was used to prepare DSPC vesicles by the hydration-extrusion procedure (explained in V. Joguparthi, T. X. Xiang, and B. D. Anderson. Liposome transport of hydrophobic drugs: gel phase lipid bilayer permeability and partitioning of the lactone form of a hydrophobic camptothecin, DB-67. J. Pharm. Sci. 97: 400-420 (2008)) at 60° C. Following preparation, vesicles were allowed to cool down for 1 h at room temperature and were subsequently dialyzed for 6 h at 37° C. against 2 L of 70 mM carbonate buffer (pH 9.5, osmolality adjusted to that of the vesicles with NaCl) to remove unentrapped HPβCD as described in V. Joguparthi and B. D. Anderson. Effect of Cyclodextrin Complexation on the Liposome Permeability of a Model Hydrophobic Weak Acid. Pharmaceutical Research 25(11):2505-2515 (2008). Following the dialysis period, vesicles were transferred to a 60° C. incubator and the outer monolayer was pegylated at 5 mol % (using m-PEG DSPE) similarly as described earlier. Following the post-pegylation procedure, vesicles were stored at 5° C. until the animal studies. One day prior to the start of the animal studies, vesicles were dialyzed overnight (37° C.) against 70 mM carbonate buffer (pH 9.5, osmolality adjusted to that of the vesicles with NaCl) and treated for animal injections similar to the vesicle preparation described above (see dosing section).

On the day of the animal studies, the entrapped concentration of DB-67 was analyzed 30 min before injection into mice.

DB-67 lactone and carboxylate plasma concentrations were analyzed by an isocratic HPLC method with fluorescence detection as described in Horn, J., et al. Validation of an HPLC method for analysis of DB-67 and its water soluble prodrug in mouse plasma. J Chromatogr B Analyt Technol Biomed Life Sci. 844, 15-22 (2006). Separation was achieved on a reverse phase C-18 column (Waters Nova-Pak, 4 μm, 3.9×150 mm) and mobile phase consisted of a mixture of 0.15 M NH₄OAc (containing 10 mM tetrabutylammonium dihydrogenphosphate (pH 6.5)) and acetonitrile (65:35, (v/v)). DB-67 lactone (1-300 ng/mL) and carboxylate (2.5-300 ng/mL) standards were prepared in mobile phase and all samples from extraction were diluted in mobile phase as required prior to analysis. The lipid. concentration of the liposome suspension dosed into animals was analyzed by HPLC method with evaporative light scattering detection (ELSD).

Due to differences in drug loading by various formulation procedures, it was not possible to precisely control the dose of drug injected into animals. Instead, the suspension lipid concentration (˜30 mg/mL) of all the formulations employed in these studies was controlled prior to animal injection. The target drug dose in these studies was 10 mg/kg. The injection volume was ˜140-150 μL per animal. The weight of the animals employed in these studies was close to each other (21-24 gm) and an average weight of 23 gm was used to estimate dose. The final drug dose administered into each animal was calculated based on the average injected volume, average animal weight, and the formulation concentration of DB-67.

Table 2 shows the final drug and lipid dose for each formulation administered into animals. The dose of DB-67 was different between the various formulations but within an order of magnitude. Therefore, the small differences in the DB-67 dose were assumed to not affect the pharmokinetics of the liposomal DB-67.

TABLE 2
Dose of liposome formulations employed
in the pharmacokinetic studies in mice

| | Lipid | DB-67 |
|---|---|---|
| Method of Loading | (mg/kg) | (mg/kg) |
| Blank vesicles spiked with DB-67 lactone | 190.7 | 6.2 |
| High intravesicular pH with HPβCD | 189.8 | 2.0 |

The pH and osmolality (freezing point depression method (Model 110 Osmometer, Fiske Associates, Norwood, Mass.)) of all the liposome formulations was monitored during each step of the formulation process. The pH and osmolality of the buffers and dialysate solutions employed used in various steps all adjusted to the pH (with 0.1 N HCl or NaOH) and osmolality (with NaCl) of the liposome suspension. The particle size of all the liposome suspensions was measured prior to the size exclusion step before animal injections by dynamic light scattering (DLS) using Malvern Zetasize-3000 (Malvern Instruments Ltd, Malvern, UK).

The pH, particle size and osmolality of all formulations were measured prior to the size exclusion performed before injection into animals. Table 3 shows the pH, particle size, and osmolality of the formulations employed in these studies.

TABLE 3
Measured pH, osmolality and particle size of
liposomes employed in pharmacokinetic studies

| | | Particle Size | Osmo- |
|---|---|---|---|
| | Final | (nm, Mean ± | lality |
| Method of Loading | pH | S.D.) | (mOsm) |
| Blank vesicles spiked with DB-67 lactone | 4.1 | 139 ± 44 | 296 |
| High intravesicular pH with HPβCD | 9.51 | 152 ± 54 | 294 |

All animal experiments were approved by the University of Kentucky Institutional Animal Care and Use Committee. Female C57BL/6 mice (Harlan, Indianapolis, Ind.) weighing between 18-24 gm were employed in these experiments. Three animals were used per time point in the pharmacokinetic studies and each animal was sampled at three to four different time points over the course of a week. Liposomal formulations were administered as a bolus into the lateral tail vein. Following administration, blood samples of approximately 75 μL were taken from the saphenous vein at 5, 30 min and 1, 1.5, 3, 6, 12, 24, 36 h and collected in heparinized microcentrifuge tubes. For the lipsomal formulation prepared by the active loading method, samples were taken at additional time points of 57 h and 72 h. The collected blood was immediately centrifuged at 1000 RPM for 5 min to separate plasma. DB-67 was extracted from plasma (by centrifugation at 1000 RPM for 5 min) using methanol stored on dry ice (plasma:methanol 1:4). Following extraction, samples were stored at −80° C. until analysis.

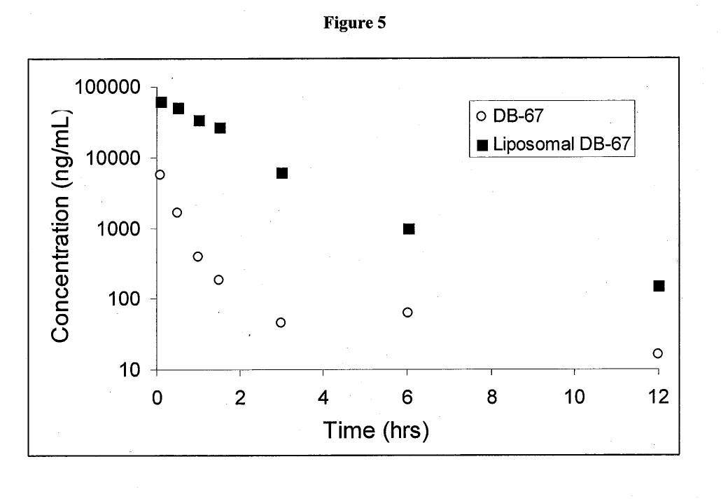

FIG. 5 shows the DB-67 plasma concentration versus time profiles of liposomes loaded with DB-67 at high intravesicular pH in the presence of cyclodextrin (▪) and blank vesicles spiked with DB-67 (∘) (i.e. non-liposomal DB-67 as the drug is outside the vesicles at the time of administration).

### Example 23

### Efficacy of DB-67 in Non-Small Cell Lung Cancer (H460) Xenografts in Mice

Non-small cell lung cancer (H460) tumor was implanted in the flank region of nu/nu mice (body weight 20-25 g). When the tumors were palpable, mice (n=7 per treatment group) were randomized to four treatment groups and received a) control (5% dextrose in water [D5W] intravenously; b) 7.5 mg/kg/day intravenously for 5 days per cycle (1 cycle=21 days); c) 3.75 mg/kg/day intravenously for 10 days per cycle; or d) 2.5 mg/kg/day intravenously for 15 days per cycle. The maximum tolerated dose (MTD) of DB-67 administered by the intravenous route was determined to be 7.5 mg/kg/day for 5 days.

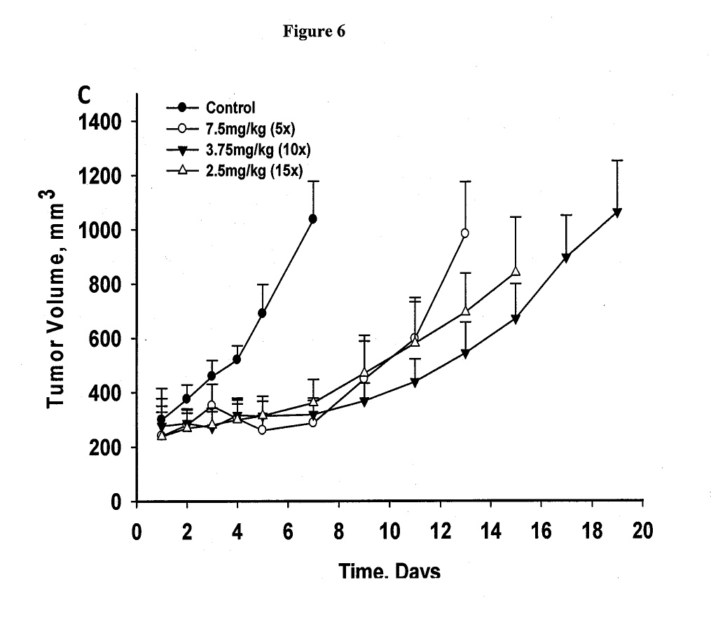

The width and length of the tumors were measured using a caliper every other day for the duration of the study. Tumor volume was calculated using the following formula:

$$V = PI ⋆ \frac{a ⋆ {b}^{2}}{6}$$

where V is tumor volume in mm³, PI=3.1416, a=size of the longest side in millimeters, and b=size of the shortest side in millimeters. Mice were euthanized when their tumor volume reached 1500 mm³ for humane reasons. FIG. 6 shows tumor volume as a function of time for the four treatment groups.

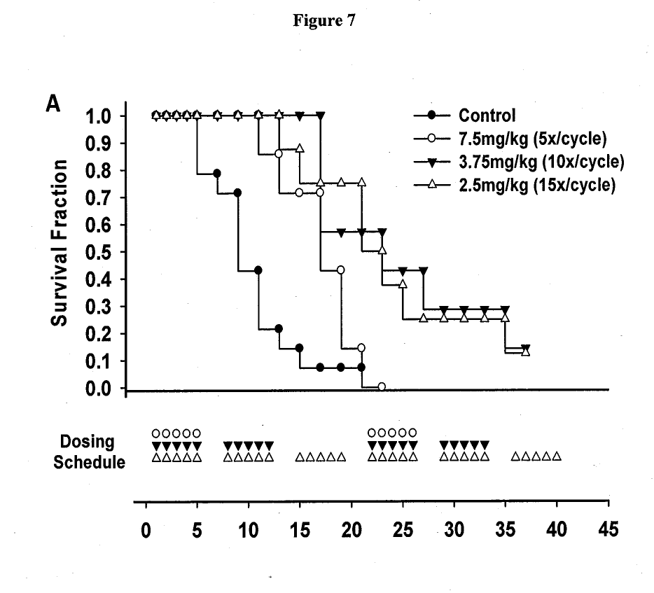

FIG. 7 is a plot showing dosing schedule for the four treatment groups and the survival fraction for the four treatment groups as a function of dosing schedule. Comparison between the median survivals of different treatment groups was done using Kaplan-Meir survival analysis.

FIGS. 6 and 7 demonstrate that protracted dosing of nonliposomal DB-67 is effective in treating non-small cell lung cancer (H460) in mice.

It will be appreciated that, although specific embodiments of the invention have been described herein for purposes of illustration, various modifications may be made without departing from the spirit and scope of the invention. All such modifications and variations are intended to be included herein within the scope of this disclosure and the present invention and protected by the following claims.

That which is claimed is:

1. A liposome comprising a hydrophobic lactone drug and a cyclodextrin, wherein the liposome has an intraliposomal pH and a cyclodextrin concentration such that upon administration of the liposome to a subject, the liposome exhibits a uniform release profile of the hydrophobic lactone drug.

2. The liposome according to claim 1, wherein the intraliposomal pH is such that the hydrophobic lactone drug is in a lactone ring-opened form.

3. The liposome according to claim 1, wherein the intraliposomal pH is such that the hydrophobic lactone drug is in the form of a ring-opened carboxylate.

4. The liposome according to claim 1, wherein the hydrophobic lactone drug is a camptothecin and the intraliposomal pH is such that the camptothecin is in the form of a ring-opened carboxylate.

5. The liposome according to claim 1, wherein the hydrophobic lactone drug is selected from the group consisting of a camptothecin, statin, parthenolide, candimine, himbacine, narcotine, hydrastine, and homolycorine.

6. The liposome according to claim 5, wherein the hydrophobic lactone drug is a camptothecin.

7. The liposome according to claim 6, wherein the camptothecin is selected from the group consisting of camptothecin, DB-67, SN-38, topotecan, irinotecan, 9-nitro-camptothecin, lurtotecan, exatecan, gimatecan, and karenitecin.

8. The liposome according to claim 7, wherein the camptothecin is DB-67.

9. The liposome according to claim 5, wherein the hydrophobic lactone drug is a statin.

10. The liposome according to claim 1, wherein the cyclodextrin is selected from the group consisting of β-cyclodextrin, analogs thereof, and derivatives thereof.

11. The liposome according to claim 10, wherein the cyclodextrin is sulfobutyl ether β-cyclodextrin or hydroxypropyl β-cyclodextrin.

12. The liposome according to claim 1, wherein the intraliposomal pH is between about 6 and about 10.

13. The liposome according to claim 1, wherein the liposome comprises a mixture of phospholipids.

14. The liposome according to claim 13, further comprising cholesterol.

15. The liposome according to claim 13, wherein the mixture of phospholipids comprises a first phospholipid selected from the group consisting of distearoylphosphatidyl choline, dipalmitoylphosphatidyl choline, diarachidonoyl phosphatidyl choline, hydrogenated soy phosphatidyl choline, dimyristoylphosphatidyl glycerol, dioleoylphosphatidylglycerol, dimyristoylphosphatidylcholine, phosphatidyl choline and phosphatidyl ethanolamine, and a second phospholipid selected from the group consisting of distearoylphosphatidic acid, hydrogenated soy phosphatidic acid, dimyristoylphosphatidic acid and phosphatidic acid.

16. The liposome according to claim 15, further comprising pegylated phospholipid.

17. The liposome of claim 1, wherein the liposome is made of unilamellar vesicles.

18. The liposome of claim 1, wherein the hydrophobic lactone drug in a lactone ring-closed form has a solubility in water less than 1 mg/ml.

19. The liposome of claim 1, wherein the total solute concentration in the aqueous compartment of the liposome is 0.4 M or less.

20. A method of administering a hydrophobic lactone drug to a subject in need thereof, comprising administering a liposome to the subject in need, wherein the liposome comprises the hydrophobic lactone drug and a cyclodextrin, the liposome having an intraliposomal pH and a cyclodextrin concentration such that upon administration of the liposome to the subject, the liposome exhibits a uniform release profile of the hydrophobic lactone drug.

21. The method of claim 20, wherein release of the hydrophobic lactone drug is prolonged relative to release of the hydrophobic lactone drug from the liposome which does not contain cyclodextrin.

22. The method of claim 20, wherein the hydrophobic lactone drug is in a lactone ring-closed form at an intraliposomal pH of 4.

23. The method of claim 20, wherein the liposome exhibits first order release kinetics.

24. The method of claim 20, wherein the hydrophobic lactone drug is a camptothecin which is used to treat cancer in the subject.

25. The method of claim 20, wherein the hydrophobic lactone drug is a statin which is used to treat high cholesterol in the subject.

26. The method of claim 20, wherein the hydrophobic lactone drug is a statin which is used to treat cancer in the subject.
